(19) United States
(12) Patent Application Publication
(10) Pub. No.: US 2016/0227781 A1
(43) Pub. Date: Aug. 11, 2016

(54) ANTIBACTERIAL TREATMENT AGENT, ANTIBACTERIAL TREATMENT METHOD, AND WATER SUPPLY-AND-DRAINAGE MEMBER

(71) Applicant: LIXIL CORPORATION, Tokyo (JP)

(72) Inventors: Youichi WATANABE, Tokyo (JP); Yasuhiro ITODA, Tokyo (JP); Yuki SHUTO, Tokyo (JP)

(21) Appl. No.: 15/023,921

(22) PCT Filed: Sep. 25, 2014
(86) PCT No.: PCT/JP2014/075513

§ 371 Date: Mar. 22, 2016

(30) Foreign Application Priority Data
Sep. 27, 2013 (JP) 2013-201342

(51) Int. Cl. A01N 55/00; B08B 17/00; A61L 2/23

(52) U.S. Cl. CPC A01N 55/00; A61L 2/23; B08B 17/00

(57) ABSTRACT

A highly hot-water-resistant antibacterial treatment agent is provided, which is capable of suppressing the sliminess of water supply-and-drainage members such as drainage members, for a long period of time, equipped to a kitchen sink or bathroom drain. The antibacterial treatment agent includes: a quaternary ammonium salt represented by Formula (1) below; and a polyhydric carboxylic acid having a C6 or higher hydrocarbon group and two or more carboxyl groups. (In Formula (1), R1 represents a C16-18 alkyl group; R2A, R2B, R4A, R4B and R4C each independently represent a C1-3 alkyl group; R3 represents a C1-4 alkylene group; and X represents a halogen atom).

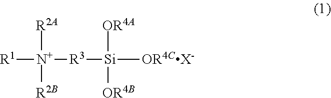

### TECHNICAL FIELD

The present invention relates to an antibacterial treatment agent, an antibacterial treatment method using the antibacterial treatment agent, and a water supply-and-drainage member that is antibacterially treated with the antibacterial treatment agent, all of which are favorably used for suppressing the sliminess of water supply-and-drainage members such as drainage members equipped to a kitchen sink or bathroom drain.

### BACKGROUND ART

Contamination components, such as foodstuff or oils and fats, attach to and remain on the surfaces of drainage members, such as a drain trap provided in a built-in kitchen sink, a sink strainer arranged over the drain trap, and a drainage plate covering the opening of the sink strainer; and as a result, bacteria grow, sliminess may occur, and odors may be generated.

Patent Document 1 proposes a treating method for suppressing the sliminess of such drainage members, in which a substrate surface of a drainage member is surface-treated with a surface-treatment agent such as mercaptopropyltrimethoxysilane, is antibacterially treated with 3-(trimethoxysilyl propyl) octadecyl dimethylammonium chloride, and is subsequently heat-treated. In this method, by heating at high temperature, a silanol group of 3-(trimethoxysilyl propyl)octadecyl dimethylammonium chloride is covalently bound to a silanol group formed on the substrate surface through the surface treatment, thereby enhancing the adhesiveness and durability in relation to the substrate surface, and realizing long-term sustainability of the effects of suppressing the sliminess.

Patent Document 1: Japanese Unexamined Patent Application, Publication No. 2011-72868

### DISCLOSURE OF THE INVENTION

### Problems to be Solved by the Invention

Antibacterially-treated surfaces, such as drainage members in a kitchen sink, are naturally required to have water resistance, and are also required to have abrasion resistance to washing with sponges or the like, chemical/alkali resistance to detergent/bleach, acid resistance to vinegar, etc., and oil resistance to edible oil.

Further, since hot water or boiling water may be drained into the drain, the antibacterially-treated surfaces are also required to have hot-water resistance.

The antibacterially-treated surface which can be formed with the method of Patent Document 1 is superior in adhesiveness, durability and chemical resistance, but is not sufficient in hot-water resistance, suffering from a problem of losing the antibacterial effects when processed with boiling water.

An object of the present invention is to provide: an antibacterial treatment agent capable of forming a highly hot-water-resistant antibacterially-treated surface; an antibacterial treatment method using the antibacterial treatment agent; and a water supply-and-drainage member which is antibacterially treated with the antibacterial treatment agent.

### Means for Solving the Problems

The inventors of the present invention have eagerly studied how to solve the problem, and have found that the problem can be solved by combining a particular polyhydric carboxylic acid with a quaternary ammonium salt that serves as an antibacterial component.

The present invention has been made based on such a finding, and is characterized in the following aspects.

A first aspect of the present invention is an antibacterial treatment agent including a quaternary ammonium salt (A) represented by Formula (1) below; and a polyhydric carboxylic acid (B) having a C6 or more hydrocarbon group and two or more carboxyl groups.

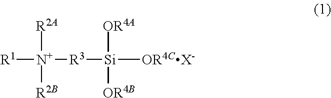

(In Formula (1), R¹ represents a C16-18 alkyl group; R^{2A}, R^{2B}, R^{4A}, R^{4B} and R^{4C} each independently represent a C1-3 alkyl group; R³ represents a C1-4 alkylene group; and X represents a halogen atom).

A second aspect of the present invention is the antibacterial treatment agent according to the first aspect, in which the quaternary ammonium salt (A) is 3-trimethoxysilyl propyl octadecyl dimethylammonium chloride.

A third aspect of the present invention is the antibacterial treatment agent according to the first or second aspect, in which the polyhydric carboxylic acid (B) is an aromatic polyhydric carboxylic acid.

A fourth aspect of the present invention is the antibacterial treatment agent according to any one of the first to third aspects, in which the antibacterial treatment agent is an aqueous solution containing the quaternary ammonium salt (A) and the polyhydric carboxylic acid (B).

A fifth aspect of the present invention is the antibacterial treatment agent according to any one of the first to fourth aspects, in which the antibacterial treatment agent contains the quaternary ammonium salt (A) and the polyhydric carboxylic acid (B), in a mole ratio represented by, quaternary ammonium salt (A):polyhydric carboxylic acid (B)=1:0.001-0.1.

A sixth aspect of the present invention is an antibacterial treatment method, wherein the antibacterial treatment agent according to any one of the first to fifth aspects is attached to a surface to be antibacterially treated, and is subsequently heat-treated.

A seventh aspect of the present invention is a water supply-and-drainage member, which is antibacterially treated by way of the antibacterial treatment agent according to any one of the first to fifth aspects.

### Effects of the Invention

According to the present invention, by using a quaternary ammonium salt (A) and a polyhydric carboxylic acid (B) in combination, a stable antibacterial film is formed by virtue of the ion bond between the quaternary ammonium salt (A) and the polyhydric carboxylic acid (B); therefore, a highly hot-water-resistant antibacterially-treated surface can be formed, which is also superior in durability such as chemical resistance and abrasion resistance.

This enhances the sustainability of antibacterial effects of water supply-and-drainage members such as drainage members in a kitchen sink or bathroom, which are exposed to hot water and boiling water; thereby making it possible to suppress the sliminess for a long period of time.

### PREFERRED MODE FOR CARRYING OUT THE INVENTION

The mode for carrying out the present invention is described below in detail.

### [Antibacterial Treatment Agent]

An antibacterial treatment agent of the present invention contains a quaternary ammonium salt (A) represented be Formula (1) below, and a polyhydric carboxylic acid (B) having a C6 or more hydrocarbon group and two or more carboxyl groups, and is preferably provided as an aqueous solution of the quaternary ammonium salt (A) and the polyhydric carboxylic acid (B).

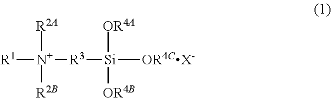

(In Formula (1), R¹represents a C16-18 alkyl group; R^{2A}, R^{2B}, R^{4A},R^{4B} and R^{4C} each independently represent a C1-3 alkyl group; R³ represents a C1-4 alkylene group; and X represents a halogen atom).

### <Quaternary Ammonium Salt (A)>

The quaternary ammonium salt (A) functions as an antibacterial component. In relation to the quaternary ammonium salt (A) represented by Formula (1) above, examples of the halogen atom represented by X include, for example, a chlorine atom and a bromine atom. In relation to the quaternary ammonium salt (A), it is preferable that 3-trimethoxysilyl propyl octadecyl dimethylammonium chloride represented by Structural Formula (la) below is used, in particular, from the perspectives of the antibacterial effects and safety.

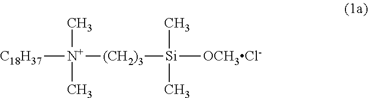

A single species of the quaternary ammonium salt (A) may be used alone, or two or more species thereof may be used in combination.

<Polyhydric carboxylic acid (B)>

A carboxyl group of the polyhydric carboxylic acid (B) is ionically bonded to a quaternary ammonium group of the quaternary ammonium salt (A) being an antibacterial component, thereby forming a stable antibacterial film. In this case, since the polyhydric carboxylic acid (B) has two or more carboxyl groups, two or more quaternary ammonium salts (A) are bonded to a single polyhydric carboxylic acid (B), thereby forming a high-molecular-weight ionically-bonded complex. This high-molecular-weight ionically-bonded complex makes it possible to form a highly hot-water-resistant and strong antibacterial film on a surface to be antibacterially treated.

In order to form a high-molecular-weight ionically-bonded complex in this manner, the number of the carboxyl groups of the polyhydric carboxylic acid (B) is required to be two or more. It is preferable that the number of the carboxyl groups of the polyhydric carboxylic acid (B) is about 2 to 4, in particular.

Further, a C5 or less hydrocarbon group of the polyhydric carboxylic acid (B) cannot form a highly hot-water-resistant antibacterially-treated surface. An upper limit of the carbon number of the hydrocarbon group is not restricted in particular, but is ordinarily 20 or below, from the perspectives of handleability and availability.

It is preferable that the hydrocarbon group of the polyhydric carboxylic acid (B) is a long-chain alkylene group, for example, a C6-12 long-chain alkylene group, in terms of the capability of forming a highly hydrophobic antibacterially-treated surface. Further, it is preferable that the aliphatic polyhydric carboxylic acid (B) having such a long-chain alkylene group has carboxyl groups at both ends of the long-chain alkylene group.

Examples of such a polyhydric carboxylic acid (B) include, for example, 1,2,3,4-butane tetracarboxylic acid, 1,10-decane dicarboxylic acid, 1,12-dodecane dicarboxylic acid, etc.

Further, it is preferable that the hydrocarbon group of the polyhydric carboxylic acid (B) is an aromatic group, in terms of the capability of forming a highly hydrophobic and structurally strong antibacterially-treated surface. It is preferable that the polyhydric carboxylic acid (B) is an aromatic polyhydric carboxylic acid, in which two or more carboxyl groups are bonded to an aromatic hydrocarbon ring, in particular. Specifically, examples of the polyhydric carboxylic acid (B) include pyromellitic acid, trimesic acid, terephthalic acid, etc., among which trimesic acid and terephthalic acid are preferable.

As long as the polyhydric carboxylic acid (B) has the carboxyl groups bonded in a radial direction as in the case of such aromatic polyhydric carboxylic acids, or as long as the polyhydric carboxylic acid (B) has carboxyl groups at both ends of the long-chain alkylene group, since the carboxyl groups are separated from each other, a quaternary ammonium salt (A) can be easily bonded thereto, and a stable high-molecular-weight ionically-bonded complex can be formed.

A single species of the polyhydric carboxylic acid (B) may be used alone, or two or more species thereof may be used in combination.

<Blend Ratio of Quaternary Ammonium Salt (A) to polyhydric carboxylic acid (B)>

In the antibacterial treatment agent of the present invention, in order to form a highly hot-water-resistant and strong antibacterially-treated surface, although the blend ratio of the quaternary ammonium salt (A) to the polyhydric carboxylic acid (B) may vary depending on the number of the carboxyl groups of the polyhydric carboxylic acid (B), it is preferable that the blend ratio is, the quaternary ammonium salt (A) to the polyhydric carboxylic acid (B)=1:0.001-0.1, and in particular, 1:0.01-0.05, in a mole ratio of the quaternary ammonium salt (A) to the carboxylic acid (B). If the quaternary ammonium salt (A) is below the range, and the polyhydric carboxylic acid (B) is above the range, the antibacterial effects tend to be deteriorated; and conversely, if the quaternary ammonium salt (A) is above the range, and the carboxylic acid (B) is below the range, the effects of enhancing the hot-water resistance to be achieved in combination with the polyhydric carboxylic acid (B) may not be sufficiently achieved.

<Aqueous Solution of Quaternary Ammonium Salt (A) and polyhydric carboxylic acid (B)>

It is preferable that the antibacterial treatment agent of the present invention is an aqueous solution of the quaternary ammonium salt (A) and the polyhydric carboxylic acid (B). Content of the quaternary ammonium salt (A) and the polyhydric carboxylic acid (B) in this aqueous solution is not limited in particular; however, excessively low content thereof deteriorates the efficiency of antibacterial treatment, and excessively high content thereof deteriorates the handleability, etc. Therefore, it is preferable that the concentration of the quaternary ammonium salt (A) in the aqueous solution when performing antibacterial treatment is about 1-5% by weight. Further, it is preferable that the concentration of the polyhydric carboxylic acid (B) in a mole ratio thereof to the quaternary ammonium salt is 1:0.001-0.1, and in particular, 1:0.01-0.05. However, the antibacterial treatment agent of the present invention may be prepared in the form of a concentrated aqueous solution having a higher concentration of the quaternary ammonium salt (A) and the polyhydric carboxylic acid (B) than the range described above, and may be diluted with water, when used. Further, an aqueous solution of the quaternary ammonium salt (A) can be mixed with an aqueous solution of the polyhydric carboxylic acid (B), when used.

### <Other Components>

The antibacterial treatment agent of the present invention may contain a quaternary ammonium salt (A) and a polyhydric carboxylic acid (B), and may contain, as necessary, other types of antibacterial components, water-soluble organic solvent such as alcohol, etc., other than the quaternary ammonium salt (A) and the polyhydric carboxylic acid (B).

However, when other components are contained in order to effectively achieve the effects of the present invention by using the quaternary ammonium salt (A) and the polyhydric carboxylic acid (B) in combination, it is preferable that the content of the other components is 10% by weight or less, in relation to the quaternary ammonium salt (A).

### [Antibacterial Treatment Method]

In an antibacterial treatment method of the present invention, the antibacterial treatment agent of the present invention is attached to a surface to be antibacterially treated, and is subsequently heat-treated.

A method for attaching the antibacterial treatment agent of the present invention to a surface to be antibacterially treated is not limited in particular; and the method may be a coating method such as spray coating; however, as an efficient method, an object to be antibacterially treated is directly immersed in the antibacterial treatment agent of the present invention, for a predetermined period of time.

If the heat treatment temperature after attaching the antibacterial treatment agent to the surface to be antibacterially treated is excessively low, a highly hot-water-resistant and highly durable antibacterially-treated surface to be achieved by the ionic bond between the quaternary ammonium salt (A) and the polyhydric carboxylic acid (B) cannot be formed; and conversely, if the heat treatment temperature is excessively high, the antibacterially-treated surface and the antibacterially-treated object may be thermally deteriorated. Therefore, the heat treatment temperature is preferably 70-150° C., and is further preferably 80-120° C. Although the heat treatment duration may vary depending on the heat treatment temperature, the heat treatment duration is preferably 0.1-2 hours, and is further preferably 0.5-1 hours.

### [Water Supply-and-Drainage Member]

A water supply-and-drainage member of the present invention is antibacterially treated with the antibacterial treatment agent of the present invention; and the antibacterial treatment method of the present invention is ordinarily applied to a method for antibacterially treating such a member.

Examples of the water supply-and-drainage members of the present invention include, for example, members as described below; however, the present invention can be widely applied to members equipped to a place around water supply and drainage, and is not limited to the following examples.

Specifically, examples of the water supply-and-drainage member of the present invention include: a drain trap, a sink strainer and a drainage plate in a built-in kitchen sink, etc.; a wash basin, a wash basin drain trap and a waste plug of a bathroom vanity; a bathroom floor, a bathroom floor drain trap and a drainage plate of a unit bath, etc.; a drain plug of a bathtub; etc.

Note that these water supply-and-drainage members are ordinarily made of metal such as stainless steel; however, application of the antibacterial treatment agent of the present invention is not limited to metal members, and can also be applied to resin members, or such members being further coated.

Further, the antibacterial treatment agent of the present invention is not limited to the use for suppressing the sliminess of water supply-and-drainage members, and can also be used as an antibacterial treatment agent for wide and general purposes.

### EXAMPLES

The present invention is described more specifically by way of the following Examples.

Note that, in the following Examples and Comparative Examples, the antibacterial effects were evaluated by measuring antibacterial activity values through the following method.

### <Method for Measuring Antibacterial Activity Value>

An antibacterial test was performed in accordance with JIS Z 2801. Escherichia coli (NBRC3972) was used as a test target bacteria. Specifically, a test piece (5 cm×5 cm) was put into a sterilized dish; 0.4 mL bacterial suspension for inoculation was inoculated into the test piece; and the top surface of the test piece was covered with a polypropylene film of 4 cm square. This was put into a desiccator, at 35° C. temperature and 90% or higher RH; and after 24 hours of contact, the number of living bacteria was measured by way of the following measuring method. Further, a bacterial suspension for inoculation was inoculated into a control piece being a non-processed film (ABS film) of the same size in substitution for the test piece; and immediately after the inoculation, and after 24 hours of contact, the number of living bacteria was measured by way of the following measuring method, in a manner similar to the test piece.

### (Method for Measuring the Number of Living Bacteria)

The polypropylene film and the test piece were put together into a sterilized polyethylene bag for stomacher; 10 mL SCDLP culture medium was added thereto; and the test bacteria were washed out by hand or stomacher. The number of living bacteria in 1 mL of this washout liquid was measured by way of an SCDLP agar medium culture method. The number of living bacteria was converted into a number per 1 cm² of the test piece.

An antibacterial activity value of each test piece was calculated in accordance with the following equation.

Antibacterial activity value=Logarithmic value of the number of living bacteria in the control piece after 24 hours−Logarithmic value of the number of living bacteria in the test piece after 24 hours

Antibacterial activity values of 2.0 or higher were evaluated as superior antibacterial effects (G); and antibacterial activity values below 2.0 were evaluated as poor antibacterial effects (P).

### Examples 1-5, Comparative Examples 1-5, and Reference Example 1

An aqueous solution was prepared, containing 3-trimethoxysilyl propyl octadecyl dimethylammonium chloride (using a methanol solution containing 3-trimethoxysilyl propyl octadecyl dimethylammonium chloride, the product name “AEM 5700”, produced by Aegis Environments Inc., in an amount of 50% by weight), and carboxylic acid shown in Table 1, in respective concentrations shown in Table 1 (however, carboxylic acid was not added to Reference Example 1); and a stainless steel plate was immersed in the aqueous solution for 30 minutes, and was pulled out. Subsequently, a heat-treatment at 90° C. was carried out for one hour. Note that, in each of Example 1-5 and Comparative Example 1-5, the mole ratio of the carboxylic acid to the 3-trimethoxysilyl propyl octadecyl dimethylammonium chloride was 0.02 mol to 1 mol. In Table 1, 3-trimethoxysilyl propyl octadecyl dimethylammonium chloride is simply described as “quaternary ammonium salt (1a)”.

In relation to each sample, a water resistance test or a hot-water resistance test was carried out as follows, and the antibacterial effects were subsequently evaluated; the result thereof is shown in Table 1.

Water resistance test 1: retained in 50° C. hot water for 16 hours

Hot-water resistance test 2: retained in 100° C. boiling water for 1 hour

Hot-water resistance test 3: retained in 100° C. boiling water for 4 hours

Note that Reference Example 1 shows antibacterial effects of a stainless steel plate without performing a water (or hot-water) resistance test.

TABLE 1

| | QUATERNARY | | | WATER (OR | | |
|---|---|---|---|---|---|---|
| | AMMONIUM SALT | CARBOXYLIC ACID | | HOT-WATER) | ANTIBACTERIAL EFFECTS | |
| | (1a) CONCENTRATION | | CONCENTRATION | RESISTANCE | ANTIBACTERIAL | EVAL- |
| | (% BY WEIGHT) | SPECIES | (% BY WEIGHT) | TEST | ACTIVITY VALUE | UATION |
| REFERENCE | 2.5% | — | — | — | 5.9 | G |
| EXAMPLE 1 | | | | | | |
| COMPARATIVE | | — | — | 50° C. [symbol] | 0.0 | P |
| EXAMPLE 1 | | | | 16 HOURS | | |
| COMPARATIVE | | ACETIC ACID | 0.12 | | 0.0 | P |
| EXAMPLE 2 | | | | | | |
| COMPARATIVE | | SUCCINIC ACID | 0.24 | | 0.0 | P |
| EXAMPLE 3 | | | | | | |
| COMPARATIVE | | CITRIC ACID | 0.38 | | 5.9 | G |
| EXAMPLE 4 | | | | | | |
| EXAMPLE 1 | | 1,12-DODECANE | 0.46 | | 5.9 | G |
| | | DICARBOXYLIC ACID | | | | |
| COMPARATIVE | | BENZOIC ACID | 0.24 | | 1.4 | P |
| EXAMPLE 5 | | | | | | |
| EXAMPLE 2 | | TEREPHTHALIC ACID | 0.33 | | 5.9 | G |
| EXAMPLE 3 | | TRIMESIC ACID | 0.42 | | 5.9 | G |
| EXAMPLE 4 | | PYROMELLITIC ACID | 0.51 | | 5.9 | G |
| COMPARATIVE | | ACETIC ACID | 0.12 | 100° C. [symbol] | 0.0 | P |
| EXAMPLE 2 | | | | 1 HOUR | | |
| COMPARATIVE | | SUCCINIC ACID | 0.24 | | 0.0 | P |
| EXAMPLE 3 | | | | | | |
| COMPARATIVE | | CITRIC ACID | 0.38 | | 0.2 | P |
| EXAMPLE 4 | | | | | | |
| EXAMPLE 1 | | 1,12-DODECANE | 0.46 | | 2.86 | G |
| | | DICARBOXYLIC ACID | | | | |
| COMPARATIVE | | BENZOIC ACID | 0.24 | | 1 | P |
| EXAMPLE 5 | | | | | | |
| EXAMPLE 2 | | TEREPHTHALIC ACID | 0.33 | | 5.9 | G |
| EXAMPLE 3 | | TRIMESIC ACID | 0.42 | | 5.9 | G |
| EXAMPLE 4 | | PYROMELLITIC ACID | 0.51 | | 4.1 | G |
| COMPARATIVE | | ACETIC ACID | 0.12 | 100° C. [symbol] | 0.0 | P |
| EXAMPLE 2 | | | | 4 HOURS | | |
| COMPARATIVE | | SUCCINIC ACID | 0.24 | | 0.0 | P |
| EXAMPLE 3 | | | | | | |
| COMPARATIVE | | CITRIC ACID | 0.38 | | 0.1 | P |
| EXAMPLE 4 | | | | | | |
| EXAMPLE 1 | | 1,12-DODECANE | 0.46 | | 0.5 | P |
| | | DICARBOXYLIC ACID | | | | |
| COMPARATIVE | | BENZOIC ACID | 0.24 | | 0.2 | P |
| EXAMPLE 5 | | | | | | |
| EXAMPLE 2 | | TEREPHTHALIC ACID | 0.33 | | 5.9 | G |
| EXAMPLE 3 | | TRIMESIC ACID | 0.42 | | 3.0 | G |
| EXAMPLE 4 | | PYROMELLITIC ACID | 0.51 | | 0.3 | P |

[symbol]

indicates data missing or illegible when filed

As is evident from Table 1, the hot-water resistance can be enhanced by using a particular polyhydric carboxylic acid in combination with 3-trimethoxysilyl propyl octadecyl dimethylammonium chloride; and in particular, in the case of using trimesic acid and terephthalic acid as the polyhydric carboxylic acid, high antibacterial effects can be maintained, even if retained in 100° C. boiling water for a long period of 4 hours.

1. An antibacterial treatment agent comprising: a quaternary ammonium salt represented by Formula (1) below; and a polyhydric carboxylic acid having a C6 or more hydrocarbon group and two or more carboxyl groups.
[Chemical Formula 1] 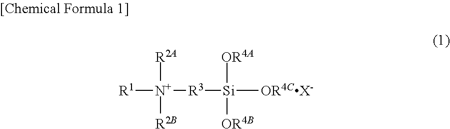
(In Formula (1), R1 represents a C16-18 alkyl group; R2A, R2B, R4A, R4B and R4C each independently represent a C1-3 alkyl group; R3 represents a C1-4 alkylene group; and X represents a halogen atom).

2. The antibacterial treatment agent according to claim 1, wherein the quaternary ammonium salt is 3-trimethoxysilyl propyl octadecyl dimethylammonium chloride.

3. The antibacterial treatment agent according to claim 1, wherein the polyhydric carboxylic acid is an aromatic polyhydric carboxylic acid.

4. The antibacterial treatment agent according to claim 1, wherein the antibacterial treatment agent is an aqueous solution containing the quaternary ammonium salt and the polyhydric carboxylic acid.

5. The antibacterial treatment agent according to claim 1, wherein the antibacterial treatment agent contains the quaternary ammonium salt and the polyhydric carboxylic acid, in a mole ratio represented by, quaternary ammonium salt:polyhydric carboxylic acid (B)=1:0.001-0.1.

6. An antibacterial treatment method, wherein the antibacterial treatment agent according to claim 1 is attached to a surface to be antibacterially treated, and is subsequently heat-treated.

7. A water supply-and-drainage member, which is antibacterially treated by way of the antibacterial treatment agent according to claim 1.
